(19) United States
(12) Patent Application Publication
(10) Pub. No.: US 2021/0369578 A1
(43) Pub. Date: Dec. 2, 2021

(54) Personal Care Compositions

(71) Applicant: Colgate-Palmolive Company, New York, NY (US)

(72) Inventors: Aida Ninfa SALINAS LOPEZ, Miguel Hidalgo (MX); Federico SANDOVAL, Ciudad de México (MX); Ramon Arnoldo LOPEZ BACA, Edo de Mex (MX); Sharon KENNEDY, Randallstown, MD (US); Alejandro JIMENEZ, Mexico City (MX)

(73) Assignee: Colgate-Palmolive Company, New York, NY (US)

(21) Appl. No.: 17/309,541

(22) PCT Filed: Dec. 7, 2018
(86) PCT No.: PCT/US2018/064405

§ 371 Date: Jun. 4, 2021

(51) Int. Cl. A61K 8/41; A61K 8/73; A61K 8/44; A61K 8/891; A61K 8/34; A61Q 5/12

(52) U.S. Cl. CPC A61K 8/416; A61K 8/737; A61K 8/44; A61K 8/891; A61K 2800/48; A61K 8/342; A61Q 5/12; A61K 2800/5426; A61K 8/731

(57) ABSTRACT

Described herein are personal care compositions that includes a carrier; a structuring agent that is ionic; and a surfactant blend comprising a zwitterionic surfactant and cetrimonium chloride.

### BACKGROUND

Human hair requires cleaning, since the hair fibers become soiled, both from environmental contamination as well as contamination and soiling from chemical agents produced by the body. Generally, shampooing is employed to clean the hair by removing excess soil and body oils which have built up on the hair fibers. Unfortunately, while being capable of cleaning the hair fibers, shampoos generally leave the hair stripped, over-processed and difficult to manage.

Hair conditioners deliver a number of desirable characteristics to hair upon treatment, including an improved wet or dry feel and a perception of softness. However, in order to secure high consumer acceptance of any hair conditioner composition, it is essential to provide consumer-desirable product aesthetics, for example not only an appealing neat product odor and a pleasant product color, but especially an appropriate product rheology and satisfactory physical product stability.

Therefore, there remains a need for hair care compositions that impart a wide range of desired performance characteristics that do not substantially interfere with each other. Some embodiments of

### BRIEF SUMMARY

The present invention is directed to a personal care composition comprising: a surfactant blend comprising: a zwitterionic surfactant having a C₁₁ alkyl group; and cetrimonium chloride; a structuring agent comprising an ionic polysaccharide; and a carrier.

In other embodiments, the present invention is directed to a personal care composition comprising: a carrier; a cationic guar gum present in an amount ranging from about 0.1 wt. % to about 0.5 wt. % based on the total weight of the personal care composition; cocamidopropyl betaine present in an amount ranging from about 5.0 wt. % to about 10.0 wt. % based on the total weight of the personal care composition; cetrimonium chloride present in an amount ranging from about 1 wt. % to about 5 wt. % based on the total weight of the personal care composition; dimethicone present in an amount ranging from about 0.5 wt. % to about 2.0 wt. % based on the total weight of the persona care composition.

In other embodiments, the present invention is directed to a method of forming a personal care composition comprising mixing together a carrier, an ionic structuring agent; and a surfactant composition comprising a zwitterionic surfactant and cetrimonium chloride to form a blend; wherein the blend has a viscosity at room temperature ranging from about 4,000 cps to about 100,000 cps.

Other embodiments of the present invention include a personal care composition comprising: a surfactant blend comprising a zwitterionic surfactant and cetrimonium chloride in a weight ratio ranging from about 2:1 to about 10:1 a structuring agent comprising an ionic polysaccharide; dimethicone; and a carrier.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Unless otherwise specified, all percentages and amounts expressed herein and elsewhere in the specification should be understood to refer to percentages by weight. The amounts given are based on the active weight of the material. According to the present application, the term “about” means+/−5% of the reference value. According to the present application, the term “substantially free” less than about 0.1 wt. % based on the total of the referenced value.

It is contemplated that the invention described herein is not limited to the particular methodology, protocols, and reagents described as these may vary. It is also to be understood that the terminology used herein is for the purpose of describing particular embodiments only and is not intended to limit the scope of the present invention in any way.

Unless defined otherwise, all technical and scientific terms used herein have the same meanings as commonly understood by one of ordinary skill in the art to which this invention belongs. All patents, patent applications, publications, and other references cited or referred to herein are incorporated by reference for all purposes.

As used herein and in the appended claims, the singular forms “a”, “an”, and “the” include plural reference unless the context clearly dictates otherwise.

The present invention relates to a composition and method of treating hair. More particularly, the present invention relates to a method of treating the hair, whereby the hair is conditioned by contacting the hair with a personal care composition (also referred to as a conditioner composition). The easy-to-apply composition is esthetically pleasing for consumer acceptance, imparts excellent wet stage and dry stage conditioner properties to the hair, and is easily rinsed from the hair. Surprisingly and unexpectedly, hair treated with the conditioner composition of the present invention also demonstrates improved physical and cosmetic properties, such as wet and dry feel and combability, softness, less coating, thickness, overall hair condition, manageability and body.

In the context of this invention, the personal care composition provides a conditioning effect to the hair treated with the conditioner composition. The term “conditioning” is to be understood for purposes of this invention as imparting positive properties to the hair, for example improved softness, enhanced shine and color brilliance, a fresh scent, and a decrease in creasing and static charge.

The personal care composition of the present invention comprises a surfactant blend. The surfactant blend comprises a first surfactant and a second surfactant. The first surfactant may comprise a zwitterionic compound. The second surfactant may comprise an ionic compound comprising a single positively (cation) or negatively (anion) charged group.

The first surfactant may be zwitterionic compound selected from one or more amphoteric surfactants having the structures:

R¹R²R³N⁺(CH₂)_{y}C(O)O⁻ (I)
or

(R¹)(R²C(O)NH(CH₂)ₓ)(R³)N⁺(CH₂)_{y}C(O)O⁻ (II)

wherein R¹ is a C₁-C₅ group which is optionally hydroxylated, such as a methyl, ethyl, hydroxyethyl, or hydroxypropyl group, R² is C₁₀-C₂₂-alkyl or C₁₀-C₂₂-alkenyl group, R³ is independently selected from a C₁-C₅ group or C₁₀-C₂₂-alkyl or C₁₀-C₂₂-alkenyl group as defined for R¹ and R², respectively, x is 2-4, and y is 2-4, and wherein any two of the groups R¹-R³ optionally may form a ring structure. C₁-C₅ group may be an alkyl or alkenyl group. Preferably, R² is C₁₀ alkyl, and R³ is independently selected from a C₁-C₅ group.

Non-limiting examples of the first surfactant include coamidopropyl betaine (“CAPB”). The first surfactant may be present in amount ranging from about 5.0 wt. % to about 10.0 wt. %—including all percentages and sub-ranges there-between—based on the total weight of the personal care composition. In some embodiments, the first surfactant may be present in amount ranging from about 6.0 wt. % to about 9.0 wt. %—including all percentages and sub-ranges there-between—based on the total weight of the personal care composition. In some embodiments, the first surfactant may be present in amount ranging from about 7.0 wt. % to about 8.0 wt. %—including all percentages and sub-ranges there-between—based on the total weight of the personal care composition. In some embodiments, the first surfactant may be present in amount of about 7.5 wt. %—based on the total weight of the personal care composition.

The second surfactant may be a quaternary ammonium salt. Non-limiting examples of the second surfactant include cetrimonium chloride (CTAC), behentrimonium chloride (BTAC), di(palmiticcarboxyethyl) hydroxyethyl methyl ammonium methylsulfate (TEP), and dimethyl di(hydrogenated tallow) ammonium chloride (DHT), N,N-bis(stearoyl-oxy-ethyl) N,N-dimethyl ammonium chloride, N,N-bis(tallowoyl-oxy-ethyl) N,N-dimethyl ammonium chloride, N,N-bis(stearoyl-oxy-ethyl)N-(2-hydroxyethyl)N-methyl ammonium methylsulfate and 1,2-di(stearoyl-oxy)-3-trimethyl ammoniumpropane chloride. In a preferred embodiment, the second surfactant is CTAC.

The second surfactant may be present in amount ranging from about 1.0 wt. % to about 5.0 wt. %—including all percentages and sub-ranges there-between—based on the total weight of the personal care composition. In some embodiments, the second surfactant may be present in amount ranging from about 1.0 wt. % to about 3.0 wt. %—including all percentages and sub-ranges there-between—based on the total weight of the personal care composition. In other embodiments, the second surfactant may be present in amount ranging from about 3.0 wt. % to about 4.0 wt. %—including all percentages and sub-ranges there-between—based on the total weight of the personal care composition. In some embodiments, the second surfactant may be present in amount of about 3.4 wt. %—based on the total weight of the personal care composition.

The first surfactant and the second surfactant may be present in the surfactant blend in a weight ratio ranging from about 1.5:1 to about 10:1—including all ratios and sub-ranges there-between. In some embodiments, the first surfactant and the second surfactant may be present in the surfactant blend in a weight ratio ranging from about 2:1 to about 10:1—including all ratios and sub-ranges there-between. In some embodiments, the first surfactant and the second surfactant may be present in the surfactant blend in a weight ratio ranging from about 2:1 to about 5:1—including all ratios and sub-ranges there-between. In some embodiments, the first surfactant and the second surfactant may be present in the surfactant blend in a weight ratio ranging from about 2:1 to about 4:1—including all ratios and sub-ranges there-between. It has been surprisingly discovered at these amounts of first and second surfactant, the resulting personal care composition provides an unexpected improvement in conditioning effects.

The personal care composition of the present invention comprises a structure agent. The structure agent imparts a structure effect to the hair treated with the conditioner composition—i.e., imparts body to the hair. The structure agent may be ionic. The structure agent may be cationic.

The structure agent may comprise a polysaccharide. The polysaccharide may be cationic. The structure agent may be selected from one or more of: cationic guar gum, cationic cellulose, cationic callose, cationic xylan, cationic mannan, cationic galactomanna n, and derivatives thereof. Cationic polysaccharides may include cationic polysaccharides that may be obtained by the use of different possible cationic etherifying agents, such as for example the family of quaternary ammonium salts.

In a non-limiting embodiment, the guar gums may be polysaccharides composed of the sugars galactose and mannose. The backbone is a linear chain of β 1,4-linked mannose residues to which galactose residues are 1,6-linked at every second mannose, forming short side-branches.

In the case of cationic polysaccharides such as cationic guars, the cationic group may be then a quaternary ammonium group bearing 3 radicals, which may be identical or different, preferably chosen from hydrogen, alkyl, hydroxyalkyl, epoxy alkyl, alkenyl, or aryl, preferably containing 1 to 22 carbon atoms, more particularly 1 to 14 and advantageously 1 to 3 carbon atoms. The counterion is generally a halogen, which is one embodiment is chlorine.

In some embodiments, the cationic polysaccharides may be selected from trimethylamino(2-hydroxyl)propyl, with a counter ion. Various counter ions can be utilized, including but not limited to halides, such as chloride, fluoride, bromide, and iodide, sulfate, methylsulfate, and mixtures thereof.

Cationic guars of the present invention may be chosen from one or more of cationic hydroxyalkyl guars, such as cationic hydroxyethyl guar (HE guar), cationic hydroxypropyl guar (HP guar), cationic hydroxybutyl guar (HB guar), and cationic carboxylalkyl guars including cationiccarboxymethyl guar (CM guar), cationic alkylcarboxy guars such as cationic carboxylpropyl guar (CP guar) and cationic carboxybutyl guar (CB guar), carboxymethylhydroxypropyl guar (CMHP guar).

The structuring agent may be present in an amount ranging from about 0.1 wt. % to about 0.5 wt. % based on the total weight of the personal care composition. In some embodiments, the structuring agent is present in an amount ranging from about 0.2 wt. % to about 0.4 wt. % based on the total weight of the personal care composition. In some embodiments directed to improved smoothness, the structuring agent is present in an amount of about 0.3 wt. % based on the total weight of the personal care composition. In some embodiments directed to improved foaming, the structuring agent is present in an amount of about 0.1 wt. % based on the total weight of the personal care composition.

The personal care composition may comprise a silicon-containing component. The silicon-containing component may comprise one or more silicone oils, such as dimethicone. Dimethicone is a mixture of fully methylated linear siloxane polymers end blocked with trimethylsiloxy units. Dimethiconol is a dimethyl silicone polymer terminated with hydroxyl groups. These silicone oils are relatively non-volatile liquids.

Silicone oils may have limited solubility in water and other polar solvents—therefore, these components are usually applied in the form of dispersions or emulsions. For example, in a water-based shampoo or personal care, the silicone oil may be dispersed with the aid of an emulsifying agent, and the dispersion or emulsion may be stabilized by the inclusion of thickeners.

The silicone oil may be copolymer, such as dimethicone-copolyol. Dimethicone-copolyol may comprise one or more copolymer of dimethylpolysiloxane with polyoxyethylene and/or polyoxypropylene side chains. The copolymer may alternatively comprise phosphated silicone polymers of the above-cited patent can be called dimethicone copolyol phosphates. In terms of chemical structure, the phosphate group forms the terminus of the polyoxyalkylene side chain, and the end hydroxyl has been converted to an ester linkage to the phosphate group.

In a non-limiting embodiment, the dimethicone copolyol may be selected from the:

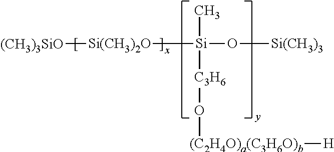

wherein x is equal to from about 3 to about 30, y is equal to from about 1 to about 10, a is equal to from 0 to about 100, and b is equal to from 0 to about 100, wherein at least one of either a or b is greater than 0 and wherein the HLB value is about 14 or less.

The dimethicone copolyols may comprise have the structure:

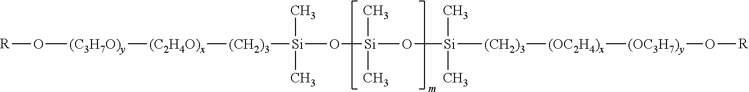

wherein the R group is hydrogen or methyl, wherein x is equal to from 0 to about 100, y is equal to from 0 to about 100, and m is equal to from about 1 to about 75, wherein at least one of either x or y is greater than 0, and wherein the HLB value is about 14 or less,

The silicon-containing component may be present in an amount ranging from 0.5 wt. % to about 2.0 wt. %—including all amounts and sub-ranges there-between—based on the total weight of the personal care composition. In some embodiments, the silicon-containing component may be present in an amount ranging from about 0.5 wt. % to about 1.5 wt. %—including all amounts and sub-ranges there-between—based on the total weight of the personal care composition. In some embodiment. In some embodiments directed to improved smoothness, the structuring agent is present in an amount of about 0.8 wt. % based on the total weight of the personal care composition. In some embodiments directed to improved foaming, the structuring agent is present in an amount of about 0.5 wt. % based on the total weight of the personal care composition.

The conditioner composition of the present invention may comprise a carrier. The carrier may be a liquid carrier. The carrier may be predominantly water, but organic solvents also can be included in order to facilitate manufacturing of the composition or to provide esthetic properties, such as viscosity control. Non-limiting examples of suitable solvents include the lower alcohols like ethyl alcohol and isopropyl alcohol; glycol ethers, like 2-butoxyethanol, ethylene glycol monoethyl ether, propylene glycol and diethylene glycol monoethyl ether or monomethyl ether; and mixtures thereof. Non-aqueous solvents can be present in the conditioner composition of the present invention in an amount of about 1% to about 50%, and in particular about 5% to about 25%, by weight of the total weight of the carrier in the composition.

The carrier may comprise water. The water may be soft water. The term “soft water” refers to water having a low concentration of ions, specifically, calcium and magnesium ions. For example, soft water is water having less than about 60 mg/l calcium carbonate, preferably less than 50 mg/l of calcium carbonate. The carrier may comprise 100 wt. % of water—specifically soft water. In other embodiments, the carrier may be predominantly water—i.e., greater than 50 wt. % water, based on the total weight of the carrier, with the remaining amounts being one or more suitable non-aqueous solvents.

The carrier may be present in an amount ranging from 70.0 wt. % to about 90.0 wt. %—including all amounts and sub-ranges there-between—based on the total weight of the personal care composition. In some embodiments, the carrier component may be present in an amount ranging from about 75 wt. % to about 85 wt. %—including all amounts and sub-ranges there-between—based on the total weight of the personal care composition. In some embodiments directed to improved smoothness, the carrier may be present in an amount of about 81 wt. % to about 83 wt. % based on the total weight of the personal care composition. In some embodiments directed to improved foaming, the carrier may be present in an amount of about 79 wt. % to about 81 wt. % based on the total weight of the personal care composition.

The conditioner composition of the present invention may further comprise one or more thickeners. The thickener may be included to improve composition esthetics and facilitate application of the conditioner composition to the hair. The thickener may be nonionic. The thickener may be present in an amount ranging from about 0.1 wt. % to about 0.5 wt. % based on the total weight of the conditioner composition. In some embodiments, the thickener may be present in an amount ranging from about 0.1 wt. % to about 0.3 wt. % based on the total weight of the conditioner composition. The thickener may be present in an amount of about 0.2 wt. % based on the total weight of the conditioner composition.

The thickener may be a water-soluble polymers and are generally water-soluble macromolecular structures having both hydrophilic and hydrophobic components. The associative polymers can thicken compositions as a result of intermolecular association between the various water-insoluble hydrophobic components which form a part of or bonded to (directly or indirectly) a water-soluble polymer backbone.

Non-limiting associative polymers suitable as a thickener may include, but are not limited to, hydrophobically modified hydroxyalkyl cellulose polymers such as hydrophobically modified hydroxyethyl cellulose (HMHEC), hydrophobically modified alkoxylated urethane polymers, such as hydrophobically modified ethoxylated urethane (HEUR), and hydrophobically modified nonionic polyols. Preferred for use herein are hydrophobically modified hydroxyalkyl cellulose polymers and mixtures thereof. In a preferred embodiment, the thickener may be cetyl hydroxyethyl cellulose.

The personal care composition may comprise one or more lipid materials. The lipid materials are essentially water-insoluble and contain hydrophobic and hydrophilic moieties. The overall amount of lipid material may range from about 0.5 wt. % to about 6 wt. %—including all amounts and subranges there-between. Lipid materials useful herein include naturally or synthetically-derived acids, acid derivatives, alcohols, esters, ethers, ketones, and amides having carbon chains of from 12 to 22, preferably from 16 to 18, carbon atoms in length. Fatty alcohols and fatty esters are preferred; fatty alcohols are particularly preferred.

Fatty alcohols included among those useful herein are disclosed in the following documents, all incorporated by reference herein: U.S. Pat. Nos. 3,155,591; 4,165,369; and 4,269,824, each of which are incorporated by reference in their entireties. Fatty esters included among those useful herein are disclosed in U.S. Pat. No. 3,341,465, hereby incorporated by reference in its entirety herein.

Preferred esters for use herein include cetyl palmitate and glycerol monostearate. Cetyl alcohol and stearyl alcohol are preferred alcohols. A particularly preferred lipid material is comprised of a mixture of stearyl alcohol and cetyl alcohol in a weight ratio ranging from about 50:50 to about 70:30—including all ratios and sub-ranges there-between.

The ester-containing lipid, for example the glyceryl monostearate, may be present in an amount ranging from may be present in an amount ranging from about 0.1 to about 1.5 wt. % based on the total weight of the personal care composition—including all percentages and sub-ranges there-between. In a preferred embodiment, the ester-containing lipid, for example the glyceryl monostearate, may be present in an amount ranging from may be present in an amount ranging from about 0.5 to about 1.0 wt. % based on the total weight of the personal care composition—including all percentages and sub-ranges there-between. In some embodiments directed to improved smoothness, the ester-containing lipid is present in an amount of about 0.5 wt. % based on the total weight of the personal care composition. In some embodiments directed to improved foaming, the ester-containing lipid is present in an amount of about 1.0 wt. % based on the total weight of the personal care composition.

The alcohol-containing lipid, for example the blend of stearyl alcohol and cetyl alcohol, may be present in an amount ranging from about 3.0 to about 6.0 wt. % based on the total weight of the personal care composition—including all amounts and sub-ranges there-between. In a preferred embodiment, the alcohol-containing lipid (e.g., the blend of stearyl alcohol and cetyl alcohol), may be present in an amount ranging from about 4.0 to about 5.0 wt. % based on the total weight of the personal care composition—including all amounts and sub-ranges there-between. In some embodiments directed to improved smoothness, the alcohol-containing lipid is present in an amount of about 4.5 wt. % based on the total weight of the personal care composition. In some embodiments directed to improved foaming, the alcohol-containing lipid is present in an amount of about 4.5 wt. % based on the total weight of the personal care composition.

The compositions herein can contain a variety of other optional components suitable for rendering such compositions more cosmetically or aesthetically acceptable or to provide them with additional usage benefits. Such conventional optional ingredients are well-known to those skilled in the art, e.g., pearlescent aids, such as mica, mother of pearl, ethylene glycol distearate preservatives and pH adjusting agents, such as citric acid, benzoic acid, benzyl alcohol, sorbic acid, methyl paraben, propyl paraben and imidazolidinyl urea. The personal care composition may further comprise coloring agents, such as any of the FD&C or D&C dyes; anti-dandruff agents, such as zinc pyrithione, selenium sulfide, coal tar, salicylic acid; hair oxidizing (bleaching) agents, such as hydrogen peroxide, perborate and persulfate salts; hair reducing agents, such as the thioglycolates; perfumes; sequestering agents, such as disodium ethylenediamine tetra-acetate. Such optional ingredients generally are used individually at levels of from about 0.01% to about 10.0%, preferably from about 0.05% to about 5.0%, of the composition.

The personal care composition of the present invention may be formed by adding together the surfactant blend, the structuring agent, and the carrier with optionally adding agitation as to mix the various components together without causing the blend to foam. The personal care composition of the present invention may be formed by adding together the silicon-containing component and lipid containing components and/or the other various components to the surfactant blend, the structuring agent, and the carrier with optionally adding agitation as to mix the various components together without causing the blend to foam.

The resulting personal care composition may have a viscosity at room temperature ranging from about 4,000 cps to about 100,000 cps—including all viscosities and sub-ranges there-between, e.g., optionally from about 10,000 cps to about 75,000 cps, further optionally from about 15,000 cps to about 50,000 cps, further optionally 20,000 cps to about 30,000 cps, further optionally from about 24,000 cps to about 25,000 cps, preferably about 24,500 cps.

### Examples

A first experiment was prepared to measure the conditioning performance of the newly formulated hair conditioning composition. Each composition was prepared using the following components:

Guar gum—cationic guar gum

Thickener—e.g. hydroxycellulose

CAPB—cocamidopropyl betaine

CTAC—cetrimonium chloride

GMS—Glyceryl monostearate

Waxy alcohols—70/30 blend of stearyl alcohol and cetyl alcohol

TABLE 1A

| | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|
| Guar Gum | 0.1 | 0.5 | 0.1 | 0.5 | 0.3 | 0.5 | 0.1 | 0.3 | 0.5 |
| Thickener | 0.1 | 0.1 | 0.1 | 0.3 | 0.1 | 0.3 | 0.1 | 0.2 | 0.2 |
| CAPB (30.5%) | 10.0 | 5.0 | 5.0 | 5.0 | 5.0 | 10.0 | 7.5 | 7.5 | 5.0 |
| CTAC (50%) | 1.0 | 3.0 | 2.0 | 3.0 | 1.0 | 2.0 | 3.0 | 2.0 | 1.0 |
| GMS | 0.5 | 0.5 | 1.0 | 1.0 | 0.5 | 0.5 | 1.0 | 0.75 | 1.0 |
| Waxy Alcohols | 4.5 | 5.0 | 5.0 | 4.5 | 4.0 | 4.0 | 4.0 | 4.5 | 4.0 |
| Dimethicone | 1.5 | 1.5 | 0.5 | 0.5 | 0.5 | 0.5 | 1.5 | 1.0 | 1.5 |
| Carrier | 82.3 | 84.4 | 86.3 | 85.2 | 88.6 | 82.2 | 82.8 | 83.75 | 86.8 |

TABLE 1B

| | Ex. 10 | Ex. 11 | Ex. 12 | Ex. 13 | Ex. 14 | Ex. 15 | Ex. 16 | Ex. 17 |
|---|---|---|---|---|---|---|---|---|
| Guar Gum | 0.1 | 0.3 | 0.1 | 0.1 | 0.5 | 0.1 | 0.5 | 0.5 |
| Thickener | 0.3 | 0.3 | 0.2 | 0.3 | 0.3 | 0.3 | 0.1 | 0.1 |
| CAPB (30.5%) | 5.0 | 10.0 | 10.0 | 5.0 | 7.5 | 10.0 | 10.0 | 10.0 |
| CTAC (50%) | 1.0 | 3.0 | 3.0 | 3.0 | 1.0 | 1.0 | 3.0 | 1.0 |
| GMS | 0.75 | 1.0 | 0.5 | 0.5 | 0.5 | 1.0 | 0.75 | 1.0 |
| Waxy Alcohols | 5.0 | 5.0 | 5.0 | 4.0 | 5.0 | 4.0 | 4.0 | 5.0 |
| Dimethicone | 1.5 | 1.5 | 1.5 | 0.5 | 1.0 | 0.5 | 0.5 | 1.0 |
| Carrier | 86.35 | 78.9 | 79.7 | 86.6 | 84.2 | 83.1 | 81.15 | 81.4 |

Each formulation was then tested on bleach hair tresses. The tests evaluated detangling wet hair, the ease of combing in wet and dry stages, as well as overall softness of hair. The individual formulations were also assessed for rheological properties, consistency, and foam formation capabilities using a Discovery Hybrid Rheometer and a Foam Shaker. The results of each test were analyzed to statistically determine relevant effects of the relevant parameter.

The detangle evaluation results are set forth in Table 2.

TABLE 2

| CAPB (30.5%) | | CTAC (50%) | | Dimethicone | |
|---|---|---|---|---|---|
| 5.0 to 7.5 | 7.5 to 10.0 | 1.0 to 2.0 | 2.0 to 3.0 | 0.5 to 1.0 | 1.0 to 1.5 |
| Decrease | Decrease | Increase | Increase | Increase | Increase |

As demonstrated by Table 2, increasing the amount of CAPB reduces (i.e., worsens) the detangle performance of the resulting hair conditioner composition. Conversely, increasing the amount of CTAC increases (i.e., improves) the detangle performance the resulting hair conditioner composition. Increasing the amount of dimethicone increases in detangle performance of the resulting hair conditioner composition.

The wet-combing evaluation results are set forth in Table 3 (below).

TABLE 3

| CTAC (50%) | | Waxy Alcohols | |
|---|---|---|---|
| 1.0 to 2.0 | 2.0 to 3.0 | 4.0 to 4.5 | 4.5 to 5.0 |
| Increase | Increase | Increase | Decrease |

As demonstrated by the data described in Table 3, increasing the amount of CTAC increases (i.e., improves) wet-combing performance of the resulting hair conditioner composition; and increasing the waxy alcohols from 4.0 to 4.5 wt. % increases (i.e., improves) the wet-combing performance of the resulting hair conditioner composition. Surprisingly, increasing the waxy alcohols from 4.5 to 5.0 wt. % decreases (i.e., worsens) the wet-combing performance of the resulting hair conditioner composition, whereby peak combing performance was achieved around 4.5 wt. %.

The dry-combing evaluation results are set forth in Table 4A.

TABLE 4A

| Guar Gum | | Thickener | | CAPB (30.5%) | | CTAC (50%) | |
|---|---|---|---|---|---|---|---|
| 0.1 to 0.3 | 0.3 to 0.5 | 0.1 to 0.2 | 0.2 to 0.3 | 5.0 to 7.5 | 7.5 to 10.0 | 1 to 2 | 2 to 3 |
| No Change | No Change | Decrease | Decrease | Decrease | Decrease | Decrease | Decrease |

As demonstrated by the data described in Table 4A, increasing the amount of guar gum increases (i.e., improves) the dry-combing performance of the resulting hair conditioner composition; increasing the amount of thickener decreases (i.e., worsens) the dry-combing performance of the resulting hair conditioner composition; increasing the amount of CAPB decreases (i.e., worsens) the dry-combing performance of the resulting hair conditioner composition; and increasing the amount of CTAC decreases (i.e., worsens) the dry-combing performance of the resulting hair conditioner composition.

TABLE 4B

| GMS | | Waxy Alcohols | | Dimethicone | |
|---|---|---|---|---|---|
| 0.5 to 0.75 | 0.75 to 1.0 | 4.0 to 4.5 | 4.5 to 5.0 | 0.5 to 1.0 | 1.0 to 1.5 |
| Increase | Increase | Decrease | Decrease | Increase | Increase |

The data described in Table 4B demonstrates that increasing the amount of GMS increases (i.e., improves) the dry-combing performance of the resulting hair conditioner composition; increasing the wax alcohols decreases (i.e., worsens) the dry-combing performance of the resulting hair conditioner composition; and increasing the amount of dimethicone increases (i.e., improves) the dry-combing performance of the resulting hair conditioner composition.

The softness evaluation results are set forth in Tables 5A and 5B.

TABLE 5A

| Guar Gum | | Thickener | | CTAC (50%) | |
|---|---|---|---|---|---|
| 0.1 to 0.3 | 0.3 to 0.5 | 1.0 to 2.0 | 1.0 to 2.0 | 5.0 to 7.5 | 7.5 to 10.0 |
| Decrease | Decrease | Decrease | Decrease | Decrease | Decrease |

As demonstrated by the data described in Table 5A, increasing the amount of guar gum decreases (i.e., worsens) the softness performance of the resulting hair conditioner composition; increasing the amount of thickener decreases (i.e., worsens) the softness performance of the resulting hair conditioner composition; and increasing the amount of CTAC decreases the softness performance (i.e., worsens) of the resulting hair conditioner composition.

TABLE 5B

| GMS | | Waxy Alcohols | |
|---|---|---|---|
| 0.5 to 0.75 | 0.75 to 1.0 | 4.0 to 4.5 | 4.5 to 5.0 |
| Increase | Increase | Increase | Increase |

The data described in Table 5B demonstrates that increasing the amount of GMS increases (i.e., improves) the softness performance of the resulting hair conditioner composition; and increasing the amount of wax alcohols increases (i.e., improves) the softness performance of the resulting hair conditioner composition.

The flash-foaming evaluation results are set forth in Tables 6A and 6B.

TABLE 6A

| Guar Gum | | Thickener | | CAPB (30.5%) | |
|---|---|---|---|---|---|
| 0.1 to 0.3 | 0.3 to 0.5 | 0.1 to 0.2 | 0.2 to 0.3 | 5.0 to 7.5 | 7.5 to 10.0 |
| Decrease | Decrease | Decrease | Decrease | Increase | Increase |

As demonstrated by the data described in Table 6A, increasing the amount of guar gum decreases (i.e., worsens) the flash-foaming performance of the resulting hair conditioner composition; increasing the amount of thickener decreases (i.e., worsens) the flash-foaming performance of the resulting hair conditioner composition; and increasing the amount of CAPB increases (i.e., improves) the flash-foaming performance of the resulting hair conditioner composition.

TABLE 6B

| GMS | | Waxy Alcohols | | Dimethicone | |
|---|---|---|---|---|---|
| 0.5 to 0.75 | 0.75 to 1.0 | 4.0 to 4.5 | 4.5 to 5.0 | 0.5 to 1.0 | 1.0 to 1.5 |
| Increase | Increase | Increase | Increase | Decrease | Increase |

The data described in Table 6B demonstrates that: a) increasing the concentration of GMS increases (i.e., improves) the flash-foaming performance of the resulting hair conditioner composition; b) increasing the waxy alcohols concentration increases (i.e., improves) flash-foaming performance of the resulting hair conditioner composition; and c) increasing the dimethicone concentration from 0.5 to 1.0 wt. % decreases (i.e., worsens) the flash-foaming performance of the resulting hair conditioner composition. Surprisingly, however, increasing the dimethicone concentration from 1.0 to 1.5 wt. % increases (i.e., improves) the flash-foaming performance of the resulting hair conditioner composition.

The max-foaming evaluation results are set forth in Tables 7A and 7B.

TABLE 7A

| Guar Gum | | CTAC (50%) | | CAPB (30.5%) | |
|---|---|---|---|---|---|
| 0.1 to 0.3 | 0.3 to 0.5 | 5.0 to 7.5 | 7.5 to 10.0 | 5.0 to 7.5 | 7.5 to 10.0 |
| Decrease | Decrease | Increase | Increase | Increase | Increase |

As demonstrated by the data described in Table 7A, increasing the amount of guar gum decreases (i.e., worsens) the max-foaming performance of the resulting hair conditioner composition; increasing the amount of CTAC increases (i.e., improves) the max-foaming performance of the resulting hair conditioner composition; and increasing the amount of CAPB increases (i.e., improves) the max-foaming performance of the resulting hair conditioner composition.

TABLE 7B

| GMS | | Waxy Alcohols | | Dimethicone | |
|---|---|---|---|---|---|
| 0.5 to 0.75 | 0.75 to 1.0 | 4.0 to 4.5 | 4.5 to 5.0 | 0.5 to 1.0 | 1.0 to 1.5 |
| Increase | Increase | Increase | Increase | Decrease | Decrease |

The data described in Table 7B demonstrates that: a) increasing the amount of GMS increases (i.e., improves) the max-foaming performance of the resulting hair conditioner composition; b) increasing the waxy alcohols increases (i.e., improves) max-foaming performance of the resulting hair conditioner composition; c) increasing the amount of dimethicone decreases (i.e., worsens) the max-foaming performance of the resulting hair conditioner composition.

Based on the above evaluations, it has been discovered that a conditioner composition set forth in Table 8 provides an unexpected improvement by balancing the following performance parameters: detangling of wet hair, the ease of combing in wet and dry stages, overall softness of hair, rheological properties, consistency, and foam formation capabilities.

TABLE 8

| Ingredient | Wt. % |
|---|---|
| Guar Gum | 0.1-0.5 |
| Thickener | 0.1-0.3 |
| CAPB (30.5%) | 5.0-10.0 |
| CTAC (50%) | 1.0-3.0 |
| GMS | 0.1-1.0 |
| Waxy Alcohols | 4.0-5.0 |
| Dimethicone | 0.5-1.5 |
| Carrier | 80.0-85.0 |

Based on the above evaluations described herein, it has been discovered that a composition as described in Table 9 provides an unexpected improvement in softness.

TABLE 9

| Ingredient | Wt. % |
|---|---|
| Guar Gum | 0.3 |
| Thickener | 0.2 |
| CAPB (30.5%) | 7.5 |
| CTAC (50%) | 3.45 |
| GMS | 0.5 |
| Waxy Alcohols | 4.5 |
| Dimethicone | 0.8 |
| Benzyl Alcohol/Benzoic Acid/Sorbic Acid | 0.3 |
| Carrier | 82.8 |

The ratio of CAPB to CTAC is about 2.2:1 and the ratio of waxy alcohols to GMS is about 9:1.

Based on the above evaluations, it has been discovered that a conditioner composition set forth in Table 10 (below) provides an unexpected improvement in foaming for the overall hair conditioner composition.

TABLE 10

| Ingredient | Wt. % |
|---|---|
| Guar Gum | 0.1 |
| Thickener | 0.1 |
| CAPB (30.5%) | 10.0 |
| CTAC (50%) | 3.45 |
| GMS | 1.0 |
| Waxy Alcohols | 4.5 |
| Dimethicone | 0.5 |
| Benzyl Alcohol/Benzoic Acid/Sorbic Acid | 0.3 |
| Perfume | 0.95 |
| Carrier | 79.4 |

The ratio of CAPB to CTAC is about 2.9:1 and the ratio of waxy alcohols to GMS is about 4.5:1.

1. A personal care composition comprising:
a surfactant blend comprising:
a zwitterionic surfactant having a C₁₁ alkyl group; and
cetrimonium chloride;
a structuring agent comprising an ionic polysaccharide; and
a cosmetically acceptable carrier.

2. The personal care composition according to claim 1, wherein the structuring agent is present in an amount ranging from about 0.1 wt. % to about 0.5 wt. % based on the total weight of the personal care composition.

3. The personal care composition according to claim 1, wherein the structuring agent comprises cationic guar gum.

4. The personal care composition according to claim 1, wherein the zwitterionic surfactant comprises cocamidopropyl betaine.

5. The personal care composition according to claim 1, wherein the zwitterionic surfactant is present in amount ranging from about 1.5 wt. % to about 3.5 wt. % based on the total weight of the personal care composition, optionally from about 1.525 wt. % to about 3.05 wt. %, further optionally 2.29 wt. %.

6. The personal care composition according to claim 1, wherein the cetrimonium chloride is present in an amount ranging from about 0.1 wt. % to about 3 wt. % based on the total weight of the personal care composition.

7. (canceled)

8. (canceled)

9. The personal care composition according to claim 1, wherein the zwitterionic surfactant ad the cetrimonium chloride are present in a weight ratio ranging from about 15:1 to about 1:1.

10.-21. (canceled)

22. A personal care composition comprising:
a carrier;
a cationic guar gum present in an amount ranging from about 0.1 wt. % to about 0.5 wt. % based on the total weight of the personal care composition;
cocamidopropyl betaine present in an amount ranging from about 1 wt. % to about 5 wt. % based on the total weight of the personal care composition;
cetrimonium chloride present in an amount ranging from about 0.1 wt. % to about 5 wt. % based on the total weight of the personal care composition;
dimethicone present in an amount ranging from about 0.5 wt. % to about 2.0 wt. % based on the total weight of the persona care composition.

23. The personal care composition according to claim 22, wherein the cetrimonium chloride is present in an amount ranging from about 0.5 wt. % to about 1.5 wt. % gr from about 3 wt. % to about 4 wt. %, based on the total weight of the personal care composition.

24. (canceled)

25. The personal care composition according to claim 22, wherein the dimethicone is present in an amount ranging from about 0.5 wt. % to about 1.5 wt. % based on the total weight of the personal care composition.

26. The personal care composition according to claim 22, wherein the carrier is present in an amount ranging from about 70 wt. % to about 90 wt. % based on the total weight of the personal care composition.

27. The personal care composition according to claim 22, further comprising cetyl hydroxycellulose present in an amount ranging from about 0.1 to about 0.3 wt. % based on the total weight of the personal care composition.

28.-30. (canceled)

31. The personal care composition according to claim 22, further comprising a blend of stearyl alcohol and cetyl alcohol, wherein the stearyl alcohol and cetyl alcohol are present in a weight ratio of about 50:50 to about 70:30, and wherein the blend of stearyl alcohol and cetyl alcohol is present in an amount ranging from about 4.0 to about 5.0 wt. % based on the total weight of the personal care composition.

32.-34. (canceled)

35. A use of a personal care composition according to claim 1, where the personal care composition is applied to the hair of a user.

36. A personal care composition comprising:
a surfactant blend comprising a zwitterionic surfactant and cetrimonium chloride in a weight ratio ranging from about 2:1 to about 10:1
a structuring agent comprising an ionic polysaccharide;
dimethicone; and
a carrier.

37. The personal care composition according to claim 36, wherein the dimethicone is present in an amount ranging from 0.5 wt. % to about 2.0 wt. % based on the total weight of the personal care composition.

38. The personal care composition according to claim 36, wherein the structuring agent is present in an amount ranging from about 0.1 wt. % to about 0.5 wt. % based on the total weight of the personal care composition, and wherein the structuring agent comprises cationic guar gum.

39. (canceled)

40. The personal care composition according to claim 36, wherein the zwitterionic surfactant comprises cocamidopropyl betaine present in amount ranging from about 1 wt. % to about 5 wt. % based on the total weight of the personal care composition.

41. (canceled)

42. The personal care composition according to claim 36, wherein the cetrimonium chloride is present in an amount ranging from about 0.1 wt. % to about 5 wt. % based on the total weight of the personal care composition.

43.-49. (canceled)

50. The personal care composition according to claim 36, further comprising a blend of stearyl alcohol and cetyl alcohol, wherein the stearyl alcohol and cetyl alcohol are present in a weight ratio of about 50:50 to about 70:30, and wherein the blend of stearyl alcohol and cetyl alcohol is present in an amount ranging from about 4.0 to about 5.0 wt. % based on the total weight of the personal care composition.

51.-53. (canceled)
